(19) United States
(12) Patent Application Publication
(10) Pub. No.: US 2009/0155289 A1
(43) Pub. Date: Jun. 18, 2009

(54) FURIN-CLEAVABLE PEPTIDE LINKERS FOR DRUG-LIGAND CONJUGATES

(76) Inventors: Steve Roberts, Potomac, MD (US); Michael S. Lebowitz, Baltimore, MD (US); Hossein A. Ghanbari, Potomac, MD (US)

Correspondence Address: M. Elisa Lane;PANACEA PHARMACEUTICALS, INC., 207 PERRY PARKWAY, SUITE 2, GAITHERSBURG, MD, 20877, US

(21) Appl. No.: 12/264,065

(22) Filed: Nov. 3, 2008

Related U.S. Application Data
(60) Provisional application No. 60/984,562, filed on Nov. 1, 2007.

(51) Int. Cl. A61K 39/395; C07K 5/00; C07K 16/18

(52) U.S. Cl. 424/178.1; 530/330; 530/391.7

(57) ABSTRACT

Disclosed are certain peptide linkers for conjugating drugs to ligands, and the resulting drug-linker-ligand molecules and compositions thereof. The conjugated molecules useful for the targeted delivery of drugs to the desired cells, and allow for the intracellular release of the drug in cases where the targeted antigen is internalized via the trans Golgi network and not the lysosomal pathway.

This application claims priority to provisional application U.S. Ser. No. 60/984,562, filed Nov. 1, 2007, the contents of which are incorporated by reference herein in its entirety.

### FIELD OF THE INVENTION

The present invention is directed to certain peptide linkers for conjugating drugs to ligands, and the resulting drug-linker-ligand molecules and compositions thereof. The invention also encompasses processes of preparation of the conjugated molecules, and methods of using them for killing or controlling the growth of cells, particularly malignant cancer cells. The peptide linkers are distinguished from known linkers in that they allow the intracellular release of the drug from the trans Golgi network.

### BACKGROUND OF THE INVENTION

Targeted delivery of cytotoxic drugs to tumor cells is desirable to avoid killing normal cells upon the systemic administration of such agents. Typical targeted drug delivery systems are composed of a cytotoxic agent conjugated to a tumor-specific antibody, forming an “immunoconjugate”. When systemically administered, the immunoconjugate will thus bind only to tumor cells in the body, and thereby deliver the cytotoxic drug intracellularly to the tumor cells, and not normal cells. The cytotoxic agent is not active when conjugated to the antibody, but will become active upon being cleaved from the antibody intracellularly.

Most endocytosed cell surface proteins are processed via the lysosomal pathway, where they are degraded by proteolysis and acidic conditions in the lysosome. Lysosome specific proteinases have thus previously been exploited to release drugs from systemically stable immunoconjugates. See, e.g., Firestone et al., U.S. Pat. No. 6,214,345. However, this strategy necessarily depends on the immunoconjugate being subject to the lysosomal pathway upon cellular internalization. Others have taken advantage of the lysosomal processing pathway in developing immunoconjugates. For example, Seattle Genetics, Inc. (Bothell, Wash., US) has developed a linker/drug technology based on specific endoproteolytic cleavage and release of MMAE and MMAF auristatins by the lysosomal proteinase cathepsin B.

Further, hydrazone bonds and stabilized disulfide bonds, which are moderately stable systemically, but labile to hydrolysis and reduction, respectively, under lysosomal conditions, have also been exploited in immunoconjugate anticancer strategies for lysosome-mediated release of highly potent calicheamicin (Wyeth's MYLOTARG™) and DM1 and DM4 maytansinoids (ImmunoGen, Inc., Waltham, Mass., US), respectively.

However, numerous plant and bacterial toxins have evolved such as to escape lysosomal degradation following cellular internalization, and to instead rely on retrograde transport through the trans Golgi network (TGN), where the specific endoproteolytic cleavage by furin will release active toxin into the cytosol, where the toxin exerts its affects by inactivating the ribosomes.

While it is known in the art that certain naturally-occurring toxins are activated intracellularly (in the TGN) by the calcium-dependent serine protease, furin, by cleavage between their protein subunits to thereby release the active toxin to the cytosol, up till now, the prior art has not taught the artificial use of a furin cleavage site to link the cell-targeting ligand component (such as an antibody or fragment thereof) to a cytotoxic small molecule drug, for the targeted delivery of the prodrug and the intracellular activation (through cleavage with furin) thereof. The present invention addresses the need for the delivery of cytotoxic drugs in cases in which a conjugated drug-ligand is internalized via the TGN (and not the lysosomal pathway).

### SUMMARY OF THE INVENTION

The present invention was developed to utilize the TGN's furin protease to release a cytotoxic drug from a drug-ligand conjugate into the cytosol, where it can exert its effects. This invention is accomplished by the insertion of an intramolecular protease cleavage site between the cytotoxic drug (i.e., a small molecule drug, rather than a proteinaceous toxin) and the cell-binding components of the targeting ligand moiety. The use of such a peptide linker thus mimics the way certain naturally-occurring toxins are activated intracellularly.

The present invention has thus been conceived to exploit the endoprotease activity and specific subcellular localization of furin in the trans Golgi network (TGN) to specifically release potent cell killing drug molecules from endocytosed immunoconjugate therapeutic agents, in cases where the internalized cell surface target receptor escapes the endosomal pathway, and thus lysosomal processing, and instead directs the bound immunoconjugate by retrograde transport to the TGN.

Thus, in one aspect of the present invention are the drug-ligand conjugates, which are linked via a furin-cleavable moiety, and pharmaceutical compositions thereof.

In other aspect, the present invention provides processes for making the drug-ligand conjugates containing the furin cleavable moiety.

In yet another aspect are methods of using the drug-ligand conjugates of the present invention to inhibit undesirable growth or activity of cells, such as cancer cells, in a subject by administering to the subject a therapeutically effective amount of the drug-ligand conjugates described herein.

### DETAILED DESCRIPTION OF THE INVENTION

The first object of the present invention is accomplished by chemically synthesizing a linker/drug molecule for chemical conjugation to a targeting ligand component, wherein the linker is composed of a peptide sequence specifically recognized and endoproteolytically cleaved by furin. The peptide sequence recognized by furin is R-X-[R/K]-R, where X is any amino acid, R is arginine, and K is lysine. The linked cytotoxic drug becomes active when released into the cytosol following furin cleavage in the TGN.

The linker/drug molecule is synthesized to also contain a bifunctional reactive component, which allows for stable chemical conjugation of the linker/drug to the targeting ligand molecule (such as an antibody or other cell surface protein/receptor-targeting molecule). An example of such a bifunctional reactive component is maleimide, which specifically reacts with free thiol groups for covalently bonding the ligand via a thioether to the drug.

The advantage of the present invention is that such conjugated “prodrugs” allow for proteolytic cleavage by furin, in the Golgi, to thereby release the active drug from a stable, specifically targeted immunoconjugate, which is for use in situations in which the cell surface target receptor for the ligand is one that escapes the typical endosomal pathway and lysosomal processing and is directed instead to the TGN. The highly specific endoproteolytic activity and specific localization of furin to the TGN enables the design of linker/drug molecules for the development of this novel immunoconjugate therapeutic strategy.

As mentioned above, most endocytosed cell surface proteins are processed via the lysosomal pathway and degraded by proteolysis and the acidic conditions in the lysosome. Lysosome-specific proteinases have thus been exploited in order to release drugs intracellularly from systemically stable immunoconjugates. However, some cell surface proteins that are specifically expressed on a target cell population, and thus highly desirable as a target for immunoconjugate or hormone prodrug therapy, escape lysosomal processing by alternative retrograde transport to the TGN.

One such cell surface protein that is an especially good target for cancer cells, and is preferred for the present invention, is the biomarker, aspartyl (asparaginyl) β-hydroxlase (AAH). For details about this cancer biomarker, see U.S. Pat. Nos. 6,783,758; 6,797,696; 6,812,206; 6,815,415; 6,835,370; and 7,094,556, the entireties of which are specifically incorporated herein by reference.

Our work on the antibody targeting of AAH and subsequate intracellular fate of the endocytosed drug-antibody indicated that processing occurs in the Golgi via the TGN, and not via the typical endosomal pathway and lysosomal processing, and thus directs the bound immunoconjugate by retrograde transport to the TGN instead. Thus, if utilizing AAH as the cellular target of an immunoconjugate (for instance), a linker as that disclosed herein, which will be cleaved by furin in the TGN, is required for activation and release of the drug moiety of the immunoconjugate into the cytosol.

The cell binding ligand component of the conjugates of the present invention is preferably a monoclonal antibody or an antigen-binding fragment thereof. More preferably, the cell binding ligand is a monoclonal antibody, or fragment thereof, that is reactive with an antigen or epitope of an antigen expressed on a cancer (whether hematopoietic or solid malignant neoplasm). The monoclonal antibody may be a murine, chimeric, humanized, or human monoclonal antibody, and may be intact, or in the form of a fragment (such as Fab, Fab′, F(ab)₂, F(ab′)₂, or single-chain Fv).

More preferably, the cell-binding ligand is an antibody, or fragment thereof, that will bind to tumor-associated biomarkers that are expressed at high levels on the target cells and that are expressed predominantly or only on diseased cells versus normal cells. Such an antibody or fragment thereof also is preferably one that will be internalized after binding to the target cell. Antibodies with such characteristics contemplated as useful for cancer-targeted conjugates of the present invention include those that target any cancer-associated antigens that are found to be internalized via the TGN, such AAH. An especially preferred embodiment in this regard are antibodies to HAAH for treating cancer in humans.

Preferably, the monoclonal antibody or fragment is human or humanized, so as to limit the possibility of an undesirable immune reaction if administered to a human patient. A humanized antibody is a recombinant protein in which the CDRs from an antibody from one species; e.g., a murine antibody, is transferred from the heavy and light variable chains of the murine antibody into human heavy and light variable domains. The constant domains of the antibody molecule are derived from those of a human antibody. Methods of humanizing non-human antibodies are known in the art, and described, for example, in U.S. Pat. Nos. 5,225,539, 5,585,089, and 5,639,641, the disclosures of which are incorporated by reference herein in their entireties. Most preferred for administration to human cancer patients is a human antibody with high specificity for and high affinity to human AAH (HAAH), which can be derived from the disclosure of U.S. Pat. No. 7,413,737, which is hereby incorporated herein in its entirety by reference.

The drug moiety useful in the linked conjugates of the present invention may be any small molecule, cytotoxic or cytostatic compounds, which are available at the present time or which are developed in the future. Most preferably, the drug is one that is particularly highly toxic in small amounts, as relatively few molecules of it will be internalized into the targeted cells (as opposed to its action systemically). Examples of such drugs are epirubicin, doxorubicin (DOX), morpholinodoxorubicin (morpholino-DOX), cyanomorpholino-doxorubicin (cyanomorpholino-DOX), 2-pyrrolino-doxorubicin (2-PDOX), MMAE and MMAF auristatins, DM1 and DM4 maytansinoids, taxol, and calicheamicin. A preferred embodiment for the drug of the conjugates of the present invention are DOX, the auristatins or the maytansinoids.

The immunoconjugate thus comprises a cell binding ligand and at least one drug for killing or inhibiting the growth of the targeted diseased cells. The cell binding agent is preferably a monoclonal antibody or a fragment thereof, and the drug moiety is preferably an anti-mitotic agent. In a particularly preferred embodiment, the immunoconjugate comprises the DOX and a human anti-HAAH monoclonal antibody. The pharmaceutical compositions of the conjugates are further comprised of a pharmaceutically acceptable carrier, excipient or diluent. A typical pharmaceutical composition of the present invention is prepared by mixing the conjugate(s) with pharmaceutically acceptable carriers, excipients or stabilizers, in the form of lyophilized formulations or aqueous solutions.

The furin-sensitive cleavage site of the conjugates of the present invention is selected from the peptide sequence R-X-[R/K]-R, where R denotes arginine, X is any amino acid, and K is lycine. The “R/K” indicates that this amino acid may be either arginine or lysine. One or more amino acids may be present in this peptide sequence for convenience during synthesis of the conjugate, as long as they do not interfere with the ultimate cleavage of the active drug component intracellelularly.

The furin-cleavage site peptide is synthetically bound to the cell-binding ligand (such as an antibody or fragment thereof), and synthetically linked at its free terminus to the small molecule drug component in such a way that the drug is stable and inactive outside of the target cell (i.e., systemically stable), until cleaved from the conjugated molecule intracellularly to its active form.

Thus, the present invention addresses a problem in the prior art concerning a way to achieve intracellular drug activation of a conjugated “prodrug” that does not enter the cell by way of the endosomal pathway, but via the TGN, in a simple yet elegant way.

More specifically, in a preferred embodiment, the drug/linker conjugate of the invention comprises 1) a maleimide group for conjugation to an AAH-targeting ligand via a highly stable thioether bond, 2) an R-X-[K/R]-R consensus recognition amino acid sequence for specific endoproteolytic cleavage by furin either following internalization and retrotransport to the trans-Golgi network or at the cell surface of AAH-expressing cancer cells, 3) a p-aminobenzylcarboxy or γ-aminobutyric acid spacer between the furin cleavage site and drug, and 4) a small molecule drug that is highly toxic to cells following its intracellular proteolytic release by furin.

The use of p-aminobenzylcarboxy or γ-aminobutyric acid spacers between the drug and furin cleavage site allows the further advantage of spontaneous hydrolytic spacer removal following enzymatic proteolysis, to give a free underivatized drug molecule.

The drug-linker-ligand conjugates of the present invention can be prepared using the reactants, conditions and synthesis schemes described in detail in U.S. Pat. No. 6,214,345 of Firestone et al. (which is hereby specifically incorporated by reference herein in its entirety), with the exception being that the peptide linker of the instant invention is different from the peptide linker of the '345 patent, requiring a modified synthesis scheme to construct our peptide.

The present invention further provides methods of treating cancer in a subject in need thereof, comprising administering to the patient a therapeutically effective amount of a conjugate described herein. The cancer to be treated is a malignant solid tumor or a hematopoietic neoplasm, and the subject is preferably a human patient.

As a preferred embodiment for the treatment of cancer in humans, the conjugate is composed of doxirubicin as drug and an anti-HAAH antibody as ligand. For such treatment, the pharmaceutical composition of this conjugate is administered parenterally in an amount of about 100 ng to about 10 mg of conjugate/kg body weight on a weekly basis during therapy.

In the further description and examples below, the abbreviations having the following meanings: R (or Arg) is arginine; K (or Lys) is lysine; T is threonine; X, X₁, and X₂ mean any amino acid, and may be the same or different; Fmoc is fluorenylmethoxycarbonyl; NHS is N-hydroxysuccinimide; DCC is dicyclohexylcarbodiimide; Mtr is 4-methoxytrtyl; EEDQ is N-ethoxycarbonyl-2-ethoxy-1,2-dihydroquinoline; MC is 6-maleimidocaproyl; PABOH is p-aminobenzyl alcohol; DOX is doxorubicin; PABC is p-aminobenzylcarbonyl; THF is tetrahydrofuran; DCU is dicyclohexylurea; Val is L-valine; DCC is dicyclohexylcarbodiimide; DME is 1,2-dimethoxyethane; MMAE is monomethylauristatin; and SSPS means solid phase peptide synthesis.

### EXAMPLES

### Example 1

### Components and Synthesis of the Peptide Linker

Essentially, the furin cleavage site peptide component of the conjugate, R-X-[R/K]-R (where X is any amino acid), is synthesized as an Mtr-blocked peptide acid by established Fmoc solid phase peptide synthesis procedures, using a hydroxymethyl-functionalized solid support resin (which allows mild acid cleavage from the resin without removing Mtr blocking groups). An Fmoc-X₂-OH group (is added N-terminally by DCC activation to the NHS ester and coupling to NH₂-R(Mtr)-X₁-K(Mtr)-OH (where X₂ is preferably K, F, R or T, but can be any natural amino acid, and X₁ is any amino acid). The C-terminal carboxylic acid is then amidated with p-aminobenzyl alcohol using EEDQ; Fmoc is removed with diethylamine; and the free amine of the N-terminal amino acid X₂ is coupled to malimidocaproyl-NHS to result in the molecule: MC-X₂-R(Mtr)-X₁-K(Mtr)-R(Mtr)-PABOH.

The PABOH group is activated with p-nitrophenol chloroformate and coupled to DOX-HCl. The Mtr blocking groups are then removed with dichloroacetic acid to result in the final drug/linker molecule, MC-X₂-R-X₁-K-R-PABC-DOX.

### Example 2

### Synthesis of the Conjugate MC-Arg-Arg-AA-Lys-Arg-PABC-DOX

In the synthesis scheme below, Arg is arginine, Lys is lysine, AA is any amino acid, and MC, PABC and DOX have the meanings given above.

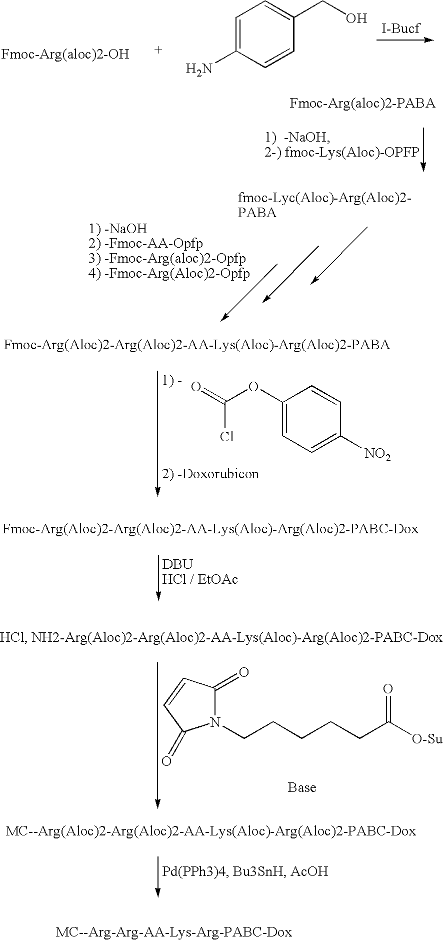

### Example 5

The immunoconjugates of the preferred embodiments of the invention are obtained by reacting the drug/furin cleavage site molecules of the above examples with the target antibody using methods well known in the art. For instance, the disulfide groups of a monoclonal antibody are reduced with dithiothreitol, and excess DTT is removed by desalting into PBS 1 mM DPTA. The reduced monoclonal antibody is reacted with 1.1 molar equivalents of the drug/linker conjugate in cold 20% acetonitrile and desalted into PBS to give the final antibody-linker-drug conjugate.

We claim:

1. A drug-linker-ligand conjugate, wherein the ligand is a molecule that specifically binds to a cell surface antigen of a targeted cell population, and wherein the linker is a furin-sensitive cleavage site peptide.

2. The conjugate of claim 1, wherein said peptide comprises R-X-[R/K]-R.

3. The conjugate of claim 1, wherein the drug is a cytotoxic, small molecule chemical, which is stably inactive extracellularly and becomes actively cytotoxic intracellularly through cleavage by furin in the Golgi of the targeted cell.

4. The conjugate of claim 1, wherein the ligand is an antibody or an antigen binding fragment thereof.

5. The conjugate of claim 1, wherein the drug is selected from epirubicin, doxorubicin (DOX), morpholinodoxorubicin (morpholino-DOX), cyanomorpholino-doxorubicin (cyanomorpholino-DOX), 2-pyrrolino-doxorubicin (2-PDOX), MMAE and MMAF auristatins, DM1 and DM4 maytansinoids, taxol, and calicheamicin.

6. The conjugate of claim 4, wherein said antibody is a monoclonal antibody.

7. The conjugate of claim 1, wherein said ligand is a murine, chimeric, humanized, or human monoclonal antibody, or antigen-binding fragments thereof.

8. The conjugate of claim 7, wherein said antibody or fragment thereof specifically binds to an antigen that is expressed on a cancer cell.

9. The conjugate of claim 8, wherein said antigen is aspartyl (asparaginyl) β-hydroxlase (AAH).

10. A method of treating a cancer in a subject, comprising administering to said subject a therapeutically effective amount of the conjugate of claim 1.

11. The method according to claim 10, wherein said cancer is a malignant solid tumor or a hematopoietic neoplasm.

12. The method of claim 11, wherein the subject is human.

13. The method of claim 12, wherein the conjugate is composed of doxirubicin as the drug, and the ligand is an anti-HAAH antibody.

14. The method of claim 13, wherein the conjugate is administered in an amount of about 100 ng to about 10 mg/kg body weight on a weekly basis during therapy.
